(19) United States
(12) Patent Application Publication
(10) Pub. No.: US 2016/0355876 A1
(43) Pub. Date: Dec. 8, 2016

(54) ASSAY DEVICES AND ASSAY ASSEMBLIES

(71) Applicant: DIAGCOR BIOSCIENCE INCORPORATION LIMITED, Kowloon Bay (CN)

(72) Inventor: Joseph TAM, Kowloon Bay (CN)

(21) Appl. No.: 15/239,431

(22) Filed: Aug. 17, 2016

Related U.S. Application Data
(63) Continuation-in-part of application No. 15/058,251, filed on Mar. 2, 2016.
Application No. 15/058,251 is a continuation of application No. 12/681,147, filed on Apr. 1, 2010, now abandoned.
(60) Provisional application No. 60/977,367, filed on Oct. 3, 2007.

(51) Int. Cl. C12Q 1/68; G01N 33/53; C12Q 1/70

(52) U.S. Cl. CPC C12Q 1/6816; C12Q 1/6834; C12Q 1/708; C12Q 1/689; C12Q 1/706; G01N 33/5302

(57) ABSTRACT

An assay assembly comprising a main housing and a capture device held inside the main housing for capturing a specific biomolecule and has a detection surface is disclosed. The main housing comprises an analyte inlet, an analyte outlet and a hybridization compartment, and the capture device is held inside the hybridization compartment with the detection surface fully extended inside the hybridization compartment. The analyte inlet is connected to an inlet end of the hybridization compartment by an analyte inlet channel and the analyte outlet is connected to an outlet end of the hybridization compartment by an analyte outlet channel. The hybridization compartment and the analyte inlet channel define a closed liquid passage path such that a liquid analyte entering the assay assembly through the analyte inlet and under a moving force or pressure will be guided to move along the analyte inlet channel, and to move through or across the hybridization compartment until subsequently discharged at the outlet end of the hybridization compartment and ultimately at the analyte outlet.

### CONTINUATION INFORMATION

This is a continuation-in-part application of U.S. Ser. No. 15/058,251 filed Mar. 2, 2016 and being abandoned.

### FIELD

The present disclosure relates to assay devices and assemblies for detection of biomolecules, and more particularly for rapid detection of biomolecules by hybridization.

### BACKGROUND

Detection of biomolecules such as nucleic acid sequences and proteins are important for many applications.

### Disclosure

An assay assembly comprising a main housing and a capture device held inside the main housing is disclosed. The capture device is for capturing one specific biomolecule or a plurality of specific biomolecule and has a detection surface, the main housing comprises an analyte inlet, an analyte outlet and a hybridization compartment, and the capture device is held inside the hybridization compartment with the detection surface fully extended inside the hybridization compartment. The analyte inlet is connected to an inlet end of the hybridization compartment by an analyte inlet channel and the analyte outlet is connected to an outlet end of the hybridization compartment by an analyte outlet channel. The hybridization compartment and the analyte inlet channel define a closed liquid passage path such that a liquid analyte entering the assay assembly through the analyte inlet and under a moving force or pressure will be guided to move along the analyte inlet channel, and to move through or across the hybridization compartment until subsequently discharged at the outlet end of the hybridization compartment and ultimately at the analyte outlet.

### FIGURES

The present disclosure is described herein by way of example and/or embodiments with reference to the accompanying figures, in which:

FIG. 1A shows an interior view of an assay assembly exposing a plurality of assay devices received inside a plurality of reaction compartments,

FIG. 1B depicts the partially exposed assay assembly of FIG. 1 with the upper half housing portions of the assay devices removed to further expose the interior of the of the assay devices,

FIG. 1C is a perspective view showing the partially exposed assay assembly of FIG. 1 with the upper half housing portion of one of the plurality of assay devices removed,

FIG. 1D is a perspective view showing the partially exposed assay assembly of FIG. 1 with all assay devices removed,

FIG. 2 is a block diagram depicting an assay apparatus according to the disclosure,

FIG. 3A is a perspective view depicting an example assay device according to the disclosure,

FIG. 3B shows the assay device of FIG. 3A with top cartridge housing removed,

FIG. 3C is a perspective cross-sectional view of the assay device of FIG. 3A along line A-A′,

FIG. 3D depicts an example assay device,

FIG. 4 depicts an example assay assembly comprising a plurality of assay devices according to the disclosure,

FIG. 4A depicts example operations of an assay device of FIG. 4,

FIG. 5A depicts an example thermal block,

FIG. 5B shows the example thermal block of FIG. 5A holding a plurality of assay assemblies,

FIG. 5C depicted a cross-sectional view of the thermal block of FIG. 5A holding a plurality of assay assemblies, and

FIG. 6A depicts example assaying operations with a plurality of assay assemblies held in a thermal block.

### DESCRIPTION

An assay assembly 100 comprises a main housing 120 and an ensemble of assay devices disposed inside the main housing 120. The ensemble of assay devices may comprise one assay device 140 or a plurality of assay devices. An analyte inlet 122, and analyte outlet 124, an ensemble of reaction compartments, and a liquid network are formed on the main housing. The ensemble of reaction compartments may comprise one reaction compartment 126 or a corresponding plurality of reaction compartments. The one assay device or the plurality of assay devices is housed within a corresponding reaction compartment or a corresponding plurality of reaction compartments formed inside the main housing.

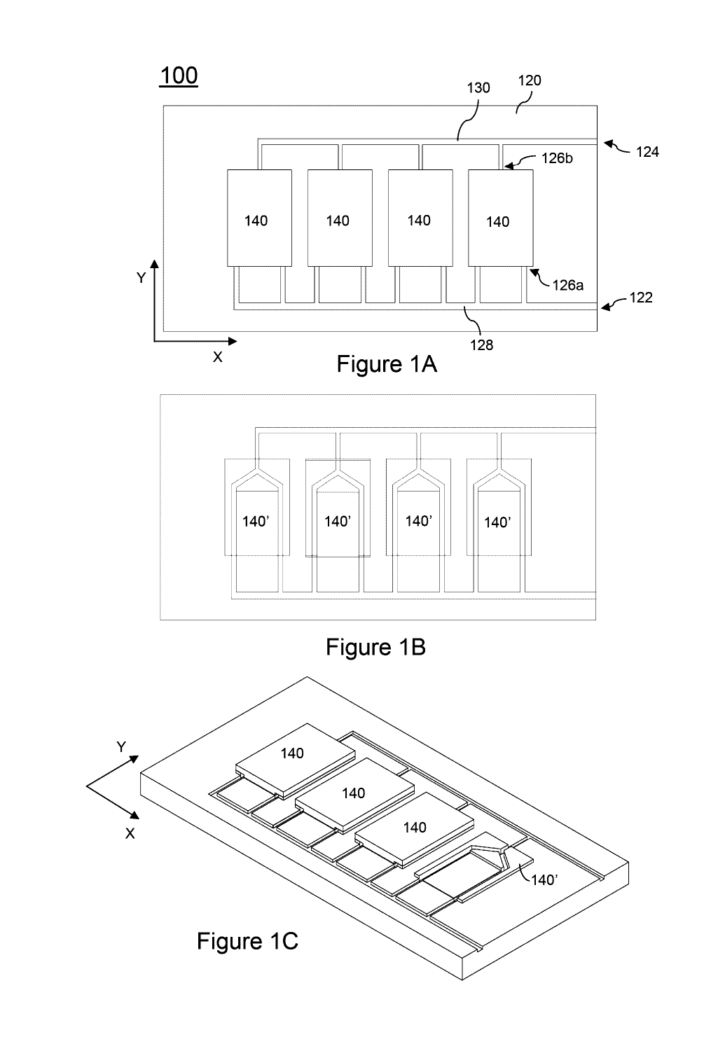

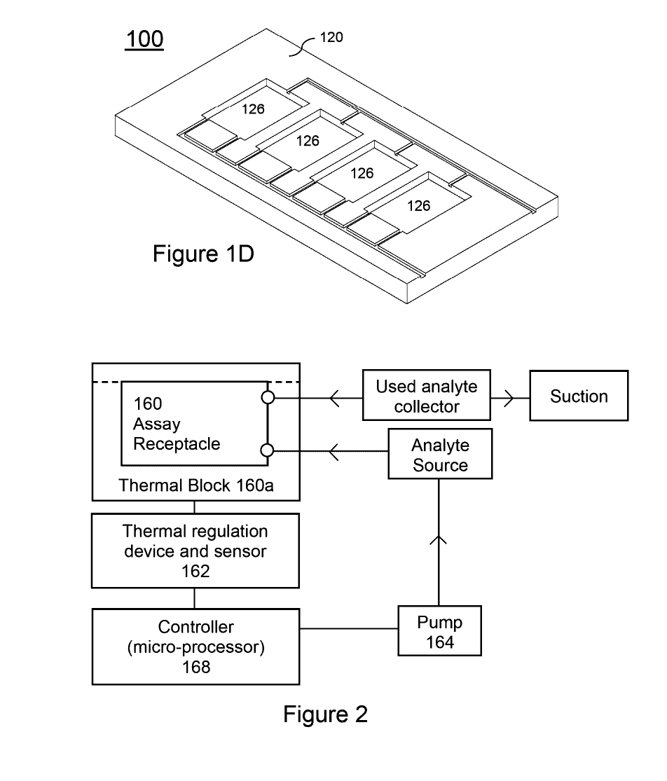

In embodiments where there is a plurality of reaction compartments connected to the same analyte inlet, such as the example depicted in FIGS. 1A to 1D, the liquid delivery channel comprises a main liquid delivery channel 128 or a liquid delivery trunk which is connected to the analyte inlet 122, and a branching channel network connecting the main liquid delivery channel to the ensemble of reaction compartments, and more particularly to an inlet end 126a of the reaction compartments. The branching channel comprises a plurality of branching channels which branches out from the main liquid delivery channel and connects with the ensemble of reaction compartments so that liquid analyte coming in from the analyte inlet will travel along the main liquid delivery channel and then the branching channel network to deliver the liquid analyte to the ensemble of reaction compartments. The reaction compartments are arranged side by side and at the same level or orthogonal distance from the main liquid delivery channel.

In embodiments where there is a single reaction compartment connected to the analyte inlet, the liquid delivery channel extends as a single, non-branching, channel between the reaction compartment and the analyte inlet.

In general, the liquid network comprises a liquid delivery channel which connects the ensemble of reaction compartments to the analyte inlet 122 and a liquid collection channel which connects the ensemble of reaction compartments to the analyte outlet 124.

In the embodiment of FIG. 1A, the liquid collection channel comprises a main liquid collection channel 130 or a liquid delivery trunk which is connected to the analyte outlet 124, and a branching channel collection network connecting the main liquid collection channel 130 to the ensemble of reaction compartments 140, and more particularly to an outlet end 126b of the reaction compartments.

To perform assaying operations, an assay assembly will be placed inside the assay receptacle of an assay apparatus. An example assay apparatus suitable to perform assaying operations in cooperation with an assay assembly according to the present disclosure may comprise an assay receptacle 160, a thermal module 162, a liquid moving module 164, and a controller module 168, and a power supply module to supply operation power. The thermal module comprises thermal conditioning device and thermal regulation device to provide thermal conditioning and thermal regulation to the assay receptacle so that assaying operations can be conducted under stable thermal conditions, for example, ±0.1° C. of a set temperature. The assay receptacle 160 may be formed on a metal block having a large thermal capacity to form a thermal block 160a to enhance thermal stability. For example, the assay receptacle 160 may be cast or molded on the thermal block. The thermal block may be made of steel, copper, aluminum, or corrosion resistant alloys. Assay receptacles may be formed as slots to dock assay assemblies. The assay receptacle may be top-loading or lateral loading. Where the assay receptacle is top-loading, the assay apparatus may comprise a top cover or other appropriate device to keep or maintain the assay receptacle under controlled operation conditions. Where the assay receptacle is lateral-loading or side loading, the assay apparatus may comprise a side door or other appropriate device to keep or maintain the assay receptacle under controlled operation conditions. The reaction compartments may be configured to receive an assay assembly to operate vertically, horizontally, at an inclination there-between, or a combination thereof where there is a plurality of reaction compartments formed in a single thermal block. Where a reaction compartment is configured to receive an assay assembly to operate vertically, the flow of target analytes may be in a direction against gravitation force, in a direction of gravitation force, or in a direction there-between, and the liquid delivery arrangements are accordingly configured. In general, the reaction compartment or the assay receptacle 160 are configured so that an assay assembly is received inside the assay receptacle 160 in a closely fitted manner or in abutment contact with the assay receptacle 160 during assaying operations. The closely fitted manner or the abutment contact between an assay assembly and the assay receptacle 160 promotes due thermal transfer contact between the assay assembly and the assay receptacle 160 and at the same time minimizing space not utilized for reaction or hybridization operations.

One or a plurality of temperature sensors is provided to monitor temperature of the assay receptacle. The temperature sensors may be embedded inside the thermal block and/or on inside surfaces of the thermal block to closely monitor temperatures. The thermal conditioning device may include heating and/or cooling devices such as heaters and coolers. The coolers may comprise a fan or fans or cooling elements such as Peltier elements. The controller may comprise a processor such as a microprocessor, ASIC (application specific circuits), logic arrays, stored or programmed with operation parameters including operation time, temperature, temperature and time profile etc. The controller may also include interfaces, for example, USB ports, WFi devices, or other connection peripheries to facilitate external control and operations so that an operator may control assaying operations remotely.

When in assaying operations, the assay assembly is placed inside the reaction receptacle of an assay apparatus, with the analyte inlet connected to a liquid analyte source for receiving liquid analyte and the analyte out connected to a liquid analyte destination for removing used or residual analyte from the assay assembly. During assaying operations, liquid analyte is moved from the liquid analyte source to the thermally controlled and regulated assay receptacle by operation of the liquid moving module. The liquid moving module may include a liquid delivery arrangement such as a pumping device and/or a suction device to facilitate moving of liquid analyte from the analyte inlet through the reaction receptacle and then to the analyte outlet. In some embodiments, the liquid delivery arrangement is to move the liquid analyte through the reaction receptacle against gravitational force. In some embodiments, the liquid delivery arrangement is to move the liquid analyte through the reaction receptacle in the direction of gravitational force.

The example assay assembly of FIG. 1 is formed into a cassette type assembly and has a rectangular or generally rectangular shape. The rectangular reaction compartments 126 are distributed along the width of the rectangular main housing at uniform spacing with the lengthwise or longitudinal sides of the reaction compartments 126 orthogonal to the lengthwise or longitudinal sides of the main housing 120. With such an arrangement, a plurality of smaller rectangular reaction compartments 126 can be formed on a single cassette housing so that assaying operations can perform simultaneously in the plurality of reaction compartments 126. The reaction compartments 126 are located at the same level relative to the lengthwise bottom of the main housing so that liquid analytes can move into the plurality of reaction compartments 126 at around the same speed and same time.

The reaction receptacle is configured to receive the assay assembly in a close fitted manner so that thermal conditions surrounding the active regions of the assay assembly are uniform or substantially uniform, for example, maintained at within ±0.05° C., ±0.1° C., ±0.2° C., ±0.3° C., etc., of a prescribed assaying temperature.

As the assay assembly 100 is substantially rectangular, the reaction receptacle includes a reaction compartment which follows or substantially follow the shape of the assay assembly and also has a rectangular or substantially rectangular shape. The reaction receptacle is disposed such that the major surfaces are vertical or substantially vertical so that the major surfaces of the assay cassette are also vertical and so that the analyte outlet is vertically above the analyte inlet during assaying operations.

In the example of FIGS. 1A to 1D, each reaction compartment has a rectangular shape and extends in a longitudinal direction Y, and the main liquid delivery channel extends in a transverse direction X which is orthogonal or substantially orthogonal to the longitudinal direction Y. The four reaction compartments are of the same shape and dimensions and are distributed in the transverse direction X at the same longitudinal level. The main liquid delivery channel extends in the transverse direction and the branching channels extend orthogonally from the main liquid delivery channel. Each reaction compartment is connected to the main liquid delivery channel by a plurality of longitudinally extending branching channels.

The main housing 120 comprises a first half and a second half which are connected together to form the analyte inlet 122, and analyte outlet 124, the reaction compartment 126 or reaction compartments and the liquid network.

In some embodiments, a first portion of the analyte inlet, the analyte outlet, the reaction compartment or reaction compartments and the liquid network is defined by the first half and a second portion of the analyte inlet, and analyte outlet, the reaction compartment or reaction compartments and the liquid network each is defined by the second half.

In some embodiments the analyte inlet, and analyte outlet may be completely formed in one of the molded halves while the reaction compartment or reaction compartments and the liquid network are cooperatively defined by the two halves.

Each of the first and second halves may be molded of hard plastics, for example, transparent plastics such as Acrylic (polymethlamethacrylate), Butyrate (cellulose acetate butyrate), Lexan (polycarbonate), and PETG (glycol modified polyethylene terphthalate) or polycarbonate. The two halves are joined to form the assay assembly, for example, by gluing, welding or other fusion methods.

When the assay assembly has been formed, a closed liquid path or circuit comprising the reaction compartment or reaction compartments and the liquid network is defined inside the main housing. With a closed liquid path formed inside the assay assembly, analyte liquid can be gradually moved upward into the reaction compartment under pressurized and controlled conditions, and fill up the reaction compartment. When the assay assembly is held in a vertical or a substantially vertical configuration such that the analyte outlet is above the analyte inlet, a liquid analyte which is driven into the main liquid delivery channel against gravity will gradually move from a bottom portion of the reaction compartment, pass through a mid-section of the reaction compartment to reach the top portion of the reaction compartment and finally exit through the analyte outlet.

During example assaying operations, the closed liquid path will function to facilitate flow of analyte liquid under controlled liquid movement conditions, for example controlled volume rate or speed, when the liquid analyte is moved by the liquid delivery arrangement.

Each assay device comprises a biomolecule capture device (or “capture device” in short) or a plurality of capture devices which is immobilized on an assay substrate. Each capture device may be a target specific capture probe which is for detection of a specific or target biomolecule. The target biomolecule may be a nuclei acid sequence, a protein sequence, or other biomolecules. The capture device may, for example, comprise a DNA probe, antibody, aptamer, or other appropriate capture probes. Biomolecules are normally not detectable in an assay system unless they are tagged for visualization or secondarily probed with another molecule that is tagged for visualization. The capture device may include a tag or label to facilitate visible identification of positive detection outcome. Fluorescence tags, quantum dot labeling, colloidal gold particle labeling, magnetic particle labeling, or enzyme-links are example techniques that may be used to facilitate visible detection of target biomolecules without loss of generality.

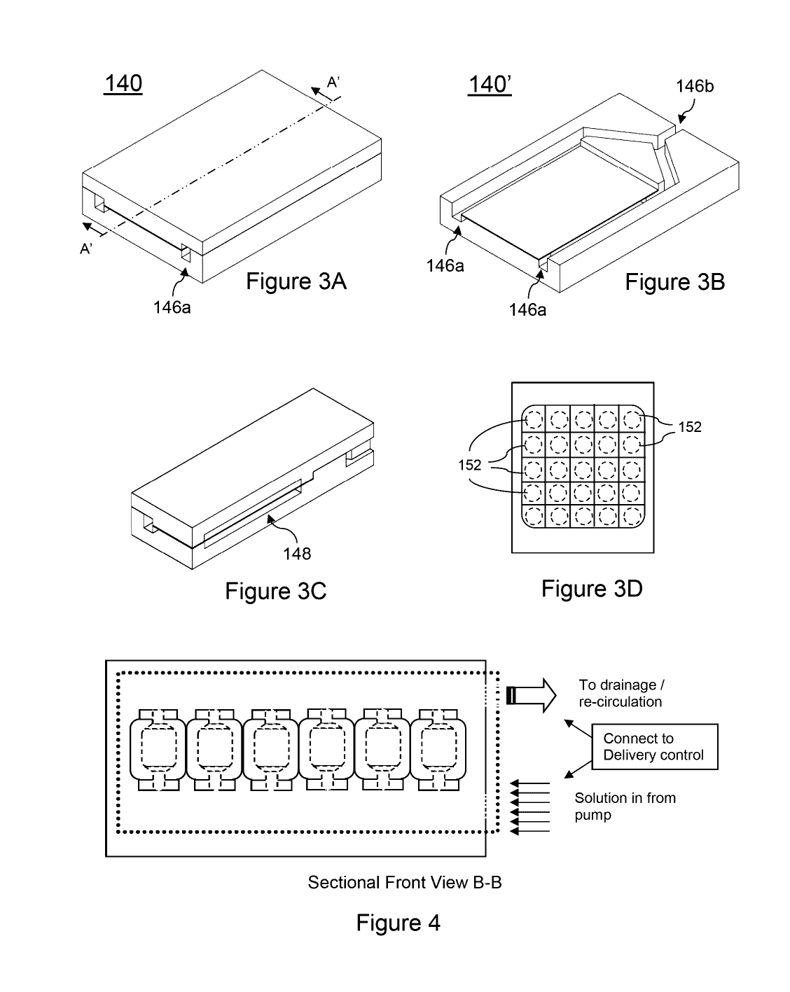

The target analyte can be any processed or suitable crude liquid samples that comprise cells, such as, but not limited to, blood, urine, sputum, and Liquid Base Cytology (LBC) samples from one or more human or animal subjects. Specifically, to be detected by said capture probes immobilized on the porous membrane, the target biomolecule may be for example a nucleic acid sequence of one or more subtypes of Human Papillomavirus (HPV) in a LBC sample; a nucleic acid sequence of any pathogens of sexually transmitted diseases in a urine sample; a nucleic acid sequence of Mycobacterium tuberculosis in a sputum sample; or an antigen against Hepatitis B virus in a blood sample.

Where a plurality of capture devices is immobilized on an assay substrate, the capture devices may be arranged in a matrix or an array as depicted in FIG. 3D. The capture device may be immobilized on a substrate, for example, a sheet type substrate such as a membrane. The membrane may be porous and made of Nylon, acetate cellulose or other suitable materials for retention of biomolecule capture devices. The pore sizes of the porous substrates may be in the region of between 3 and 8 μm, for example, around 4, 4.5 or 5 μm.

An example assay device is in the form of an assay cartridge depicted in FIG. 3A. The example assay cartridge comprises a first cartridge housing, a second cartridge housing, and a detection member. The detection member is a porous membrane on which one or a plurality of capture devices has been immobilized to form a set of biomolecule specific capture devices. The first cartridge housing defines a first major surface of the cartridge housing and the second cartridge housing defines a second major surface of the cartridge housing. The detection member is held by cooperation between the first and second cartridge housings when the first and second cartridge housings are connected to form a main cartridge housing, and when the detection member is so held or sandwiched between the first and second cartridge housings, the detection member is substantially extended with its major surface parallel to or substantially parallel to the first major surface and/or the second major surface of the cartridge housing. When the first and second cartridge housings are connected to form a main cartridge housing, a hybridization chamber is defined between the first and second cartridge housings and interior clearance of the cavity defining the hybridization chamber has a depth or thickness which is slightly larger than the thickness of the membrane member forming the detection member to maximize utilization of liquid analyte. In general, the clearance between the detection surface of the membrane member is comparable to or slightly smaller than the thickness of the membrane member, which has a typical thickness of between 150 μm to 250 μm. The example assay cartridge has an example length of 2.5 cm and a width of 1.5 cm. The thickness of the cartridge is about 0.3 to 0.5 cm.

The cartridge or the cartridge housing has a shape which is fully or substantially fully complementary to the shape of the reaction compartment 126 so that when the cartridge or the cartridge housing is received inside the reaction compartment 126, the cartridge or the cartridge is closely fitted inside the reaction compartment 126 with no or almost no space left between the cartridge or the cartridge housing and the receiving reaction compartment 126. With such an arrangement, liquid analyte coming into the reaction compartment 126 will be diverted to flow into the interior of the cartridge or the cartridge housing and to encounter the detection member therein. When the liquid analyte encounters the capture device 152 under assaying conditions, hybridization between the biomolecule specific capture device 152 and the target biomolecule and positive identification can be visually distinguished due to the presence of the labelling agents.

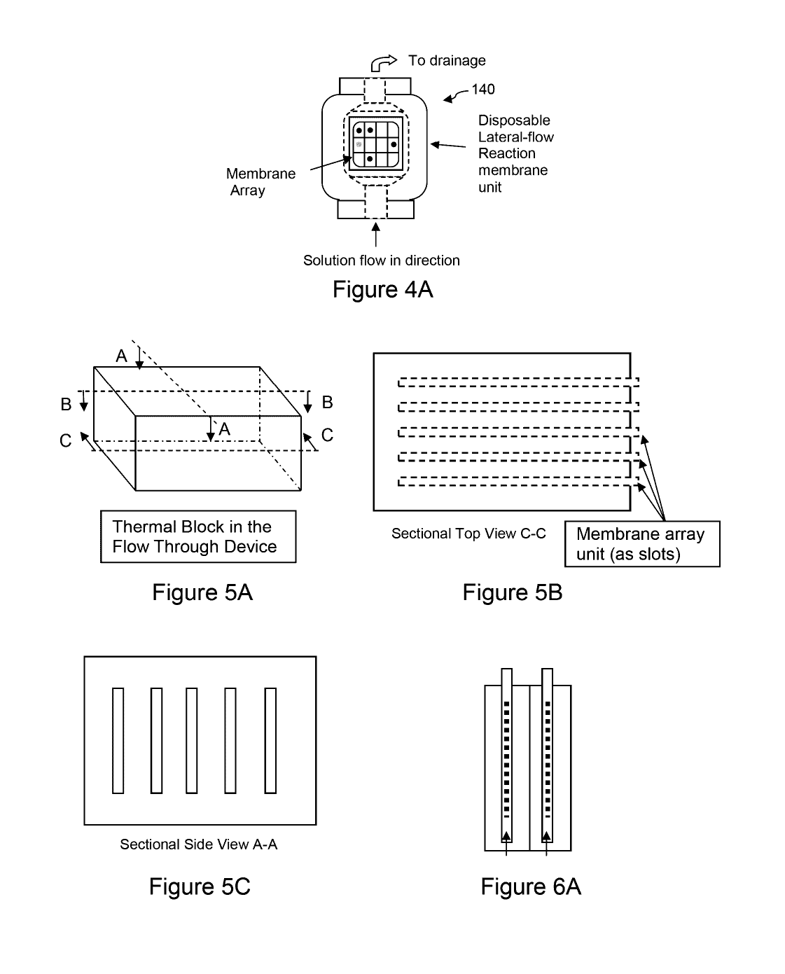

Referring to FIGS. 3B and 3C, a cartridge half comprises a base portion having a rectangular outline and defining an internal recess. The internal cavity is defined by a bed portion which is surrounded by a peripheral wall. The peripheral wall includes a bottom wall portion which extends transversely and on which a cartridge inlet 146a is formed. A pair of side wall portions extends away from the bottom wall portion in the longitudinal direction. The separation distance between the oppositely facing side wall ports define width of the internal recess. The longitudinal extent of the parallel side wall portions defines the length of the internal recess. The top wall portions tapers from the end of the parallel side wall portions towards the longitudinal center axis and merge at a longitudinal center axis. Side channels are formed between the bed portion and parallel side wall portions as liquid guides inside the cartridge to be formed. The bed portion is elevated from the bottom of the side channels and the detection member sits on the bed portion. When the two halves are joined together, the counterpart recesses cooperate to form a hybridization chamber 148 with the detection member rest squarely on the bed portion and sides of the detection member fixedly mounted by corresponding engaging side wall portion of the counterpart cartridge halves. When the cartridge is formed, most empty or cavity space of the hybridization chamber 148 is occupied by the detection membrane so that target analyte entering the cartridge will be very efficiently used. The inclined top wall portions merge at the longitudinal axis to define a cartridge outlet 146b. During assaying operations, liquid analyte entering the cartridge inlet 146a will move upwardly through the hybridization chamber 148 and encounter the capture device 152 as a detection device on the detection member. A target biomolecule upon encountering a matching target biomolecule capture probe will be captured on hybridization and the outcome visible, as depicted schematically in FIG. 4A.

An assay assembly depicted in FIG. 4 has configurations substantially identical to that of FIG. 1, except that a plurality of liquid channels is independently connected to a corresponding plurality of reaction compartments, or assay devices or hybridization chambers.

In some embodiments, the detection member or detection membrane(s) of the assay device may be directly attached to or mounted on the main housing of the assay assembly without a cartridge housing intermediate of the main housing and the detection membrane. In such embodiments, the detection member is tightly held by cooperation of the components of the main housing when the components are connected to find the main housing. Where the main housing is transparent, results and outcomes would be readily apparent from the outside of the assay assembly where the detection surfaces of the detection member are oppositely facing a viewing surface of the assay assembly.

In some embodiments, the space inside the assay assembly or reaction compartment which available for passage of target analyte is mostly occupied by the detection membrane to minimize amount of target analyte required and so that more tests can be performed using a same volume of target analyte. In example embodiments, the detection membrane may occupy 20% or more or less, 30% or more or less, 40% or more or less, 50% or more or less, 60% or more or less, 70% or more or less, or a range formed by a combination of any of the aforesaid values of the total volume inside the assay assembly or reaction compartment available for passage of target analyte.

An example thermal block depicted in FIG. 5A comprises a plurality of reaction compartments which are parallel disposed. The reaction compartment is elongate and extends along a longitudinal direction of the thermal block and projects from a lateral side of the thermal block (as depicted in FIG. 5B) for liquid coupling with the liquid delivery arrangement of the assay apparatus. The portions of the assembly assemblies not protruding from the thermal block are embedded inside the thermal block as depicted in FIG. 5C to provide a thermally stable and well-regulated assaying environment to facilitate multiple hybridization reactions at the same time using a single assaying apparatus.

An example thermal block depicted in FIG. 6A comprises a plurality of reaction compartments which are parallel disposed, but with the top portions of the assaying assemblies projecting from the top of the thermal block. During assaying operations, liquid analyte is delivered to bottom of the reaction compartment and then moved upwardly through the vertically disposed reaction compartment and traverse the detection membrane. As the liquid analyte is moved upwards against gravity and the speed is controlled, the liquid analyte will move upwards at the same rate at the plurality of assay assemblies to promote homogenous hybridization. Used analyte will exit at the top end of the assay assemblies or the reaction compartment and removed for recirculation by the liquid delivery arrangement or for disposal.

During assaying operations, the assay apparatus is powered on, and the controller will set the operation conditions and parameters according to instructions, for example stored or entered instructions. When the reaction compartment, a reservoir containing a target analyte, and other components along the liquid flow path are at the set operation conditions, the liquid delivery arrangement, for example, under operational control of the controller, will deliver the target liquid analyte from the analyte reservoir to the reaction compartment where the target analyte will encounter the detection devices and hybridize therewith when there is a matching capture. In example operations, the target analyte will stay or incubate in the reaction compartment or hybridization chamber for a preset time, for example 1 minute or two minutes before moving out. In some example operations, the target analyte is moved upwardly through the reaction compartment gradually and then stays for an incubation time to ensure due hybridization with good efficiency. Where the detection member is porous, capture probes will penetrate into the interior of the porous member and this would enhance detection efficiency as well as outcome visibility. The reverse flow against gravitation force further ensures thorough hybridization inside and outside the membrane. For example, where a porous assay member has a thickness D1, a detection member may have capture probe molecules penetrated into the porous assay member so that the capture probe molecules are on the outside surface as well as immediately underneath the capture probe molecules which are on the outside surface. Where the penetration is substantial, for example, more that 10%, more than 20%, more that 30%, more than 40%, more than 50%, more than 60%, or in a range defined by any of the aforesaid, into the thickness D1 of the porous assay member, hybridization will occur at the surface as well as inside of the porous assay member and substantial enhancement in detection efficiency will result. The lateral flow of liquid analyte through the porous assay member has demonstrated advantages over assaying by transverse flow of analyte through an assaying member. The term “lateral flow” herein means liquid analyte is to travel across the porous assay member by moving inside the assay member and between the top and bottom major surfaces of the porous assay member, and usually in a longitudinal direction. A capture device is deposited or formed on a detection surface which is usually one of the top and bottom major surfaces so that assaying outcomes are visually detectable on the detection surface. A transverse flow herein is one which is in a direction orthogonal or substantially orthogonal to the lateral flow.

While the present disclosure has been described with reference to example, embodiments and figures herein, it should be appreciated that the example, embodiments and figures are non-limiting examples and should not be used to limit the scope of disclosure.

1. An assay assembly comprising a main housing and a capture device held inside the main housing, wherein the capture device is for capturing one specific biomolecule or a plurality of specific biomolecule and has a detection surface, the main housing comprises an analyte inlet, an analyte outlet and a hybridization compartment, and the capture device is held inside the hybridization compartment with the detection surface fully extended inside the hybridization compartment; wherein the analyte inlet is connected to an inlet end of the hybridization compartment by an analyte inlet channel and the analyte outlet is connected to an outlet end of the hybridization compartment by an analyte outlet channel; and wherein the hybridization compartment and the analyte inlet channel define a closed liquid passage path such that a liquid analyte entering the assay assembly through the analyte inlet and under a moving force or pressure will be guided to move along the analyte inlet channel, and to move through or across the hybridization compartment until subsequently discharged at the outlet end of the hybridization compartment and ultimately at the analyte outlet.

2. The assay assembly according to claim 1, wherein the hybridization compartment extends in a longitudinal direction and the inlet end and the outlet end of the hybridization compartment are at opposite longitudinal ends of the hybridization compartment.

3. The assay assembly according to claim 2, wherein the analyte outlet is above the hybridization compartment and the analyte inlet is below the hybridization compartment during assaying operations.

4. The assay assembly according to claim 2, wherein the analyte inlet channel extends transversely from the analyte inlet into the main housing and longitudinally towards the hybridization compartment.

5. The assay assembly according to claim 1, wherein the capture device comprises a target specific capture probe which is for detection of a biomolecule, and the biomolecule is a nuclei acid sequence, a peptide sequence, or other biomolecules.

6. The assay assembly according to claim 5, wherein the capture device comprises a DNA probe, an aptamer, or an antibody.

7. The assay assembly according to claim 1, wherein the capture device comprises a membrane on which a target specific capture probe or a plurality of target specific probes is immobilized on the detection surface, and the detection surface is substantially planar and extended along both a longitudinal direction and a transverse direction and parallel to and/or oppositely facing a major surface of the hybridization compartment to facilitate visual observation of assay operation outcomes or results.

8. The assay assembly according to claim 1, wherein the main housing comprises a first molded half and a second molded half which are attached together to cooperatively define the inlet channel and the hybridization compartment; and wherein the capture device is restrained inside the main housing with the detection surface extended taut or near taut between opposite longitudinal ends of the hybridization compartment.

9. The assay assembly according to claim 1, wherein the capture device is mounted on a cartridge housing to form an assay cartridge and the assay cartridge is received inside the hybridization compartment.

10. The assay assembly according to claim 9, wherein the assay cartridge comprises a cartridge inlet which is connected in abutment with the analyte inlet channel, a cartridge outlet which is in liquid communication with the analyte outlet, and a hybridization chamber interconnecting the cartridge inlet and the cartridge outlet and extending in the longitudinal direction; and wherein the capture device is extended taut or near taut inside the hybridization chamber.

11. The assay assembly according to claim 10, wherein the cartridge housing forms a closed liquid passage path extending between the cartridge inlet and the cartridge outlet such that liquid analyte entering the assay cartridge through the cartridge inlet must move through the hybridization chamber before exiting at the cartridge outlet.

12. The assay assembly according to claim 10, wherein the capture device is extended such that its major surface is substantially parallel to and/or oppositely facing a major surface of the hybridization chamber and the capture probe or the plurality of capture probes is oppositely facing the major surface of the hybridization chamber to facilitate visual observation of assay or detection outcome or results after use.

13. The assay assembly according to claim 10, wherein the capture device is disposed such that one or both of its major surfaces is in abutment contact or in close proximity to a portion of the cartridge housing defining the hybridization chamber.

14. The assay assembly according to claim 10, wherein a first inlet aperture and a second inlet aperture are formed on opposite lateral sides on a bottom end of the hybridization chamber, and the hybridization chamber comprises a liquid guide to guide movement of liquid from the cartridge inlet to pass through the hybridization chamber and then merge to exit at the cartridge outlet.

15. The assay assembly according to claim 1, wherein the capture device is a porous substrate or a non-porous substrate such as membrane or paper substrate, and the thickness of the hybridization chamber is comparable to or slightly larger than the thickness of the capture device so that a very substantial portion of target analytes moving through the hybridization chamber is to encounter the capture device and interact with the capture probe or probes before leaving the hybridization chamber; and/or wherein clearance between the major surface of the capture device and the hybridization chamber is comparable to or slightly smaller than the thickness of the probe substrate.

16. The assay assembly according to claim 1, wherein a plurality of hybridization compartments is formed on the main housing, and the analyte inlet channel comprises a transversely extending main transverse channel which connects the analyte inlet to the plurality of hybridization compartments by means of a branching channel network, the branching channel network comprising a plurality of branch channels which branches from the main transverse channel and extends upwardly in the longitudinal direction to connect with the hybridization compartments.

17. The assay assembly according to claim 16, wherein the hybridization compartments are distributed in a lateral direction transverse to the longitudinal direction and are aligned at same longitudinal level.
